(19) United States
(12) Patent Application Publication
(10) Pub. No.: US 2018/0353523 A1
(43) Pub. Date: Dec. 13, 2018

(54) CONJOINT THERAPIES WITH INHIBITORS OF GLUCOSE PRODUCTION

(71) Applicant: Viking Therapeutics, San Diego, CA (US)

(72) Inventor: Brian Lian, San Diego, CA (US)

(21) Appl. No.: 15/761,914

(22) PCT Filed: Sep. 21, 2016
(86) PCT No.: PCT/US16/52842

§ 371 Date: Mar. 21, 2018

Related U.S. Application Data
(60) Provisional application No. 62/221,991, filed on Sep. 22, 2015.

(51) Int. Cl. A61K 31/675; A61P 3/00; A61K 31/155

(52) U.S. Cl. CPC A61K 31/675; A61P 3/00; A61K 31/506; A61K 2300/00; A61K 31/155

(57) ABSTRACT

The present invention provides pharmaceutical compositions and methods for treating and/or preventing a metabolic condition.

### RELATED APPLICATIONS

This application claims the benefit of priority to U.S. Provisional Patent Application Ser. No. 62/221,991, filed Sep. 22, 2015. This application is hereby incorporated by reference in its entirety.

### BACKGROUND OF THE INVENTION

Diabetes mellitus is a group of metabolic diseases where a subject has high blood glucose levels over a prolonged period. Symptoms of high blood glucose include frequent urination, increased thirst, and increased hunger. If left untreated, diabetes can cause many complications including diabetic ketoacidosis and diabetic coma. Serious long-term complications include cardiovascular disease, stroke, chronic kidney failure, foot ulcers, and damage to the eyes.

The three types of diabetes mellitus include Type 1, Type 2, and gestational diabetes. Type 1 diabetes, previously referred to as insulin-dependent diabetes mellitus or juvenile diabetes, results from the pancreas' failure to produce sufficient insulin. Type 2 diabetes, previously referred to as noninsulin-dependent diabetes mellitus or adult-onset diabetes, results from insulin resistance where cells fail to respond to insulin properly. Gestational diabetes occurs when pregnant women without a previous history of diabetes develop a high blood glucose level.

Treatments to control blood glucose levels for Type 1 diabetics involve delivery of insulin via injection or pump while the recommended initial therapy for Type 2 diabetics to control blood glucose often involves changes to diet and recommendations to increase physical activities and exercise. In addition, noninsulin oral antidiabetic drugs such as metformin have been shown to be effective to treat diabetes, particularly Type 2 diabetics with normal kidney function. However, the adverse effects of certain antidiabetic drugs, including diarrhea, cramps, nausea, vomiting, and increased flatulence, lactic acidosis, weight gain, fluid retention and impaired liver or kidney function, have limited their use.

There is a need for improved methods for treating and/or preventing metabolic conditions, such as diabetes.

### SUMMARY OF INVENTION

In one aspect, the invention relates to a pharmaceutical composition comprising an inhibitor of gluconeogenesis, or a salt, ester, or prodrug thereof; and at least one agent that promotes lactic acid clearance.

In certain embodiments, the inhibitor is an inhibitor of fructose-1,6-bisphosphatase. In certain embodiments, the inhibitor is a compound of Formula I

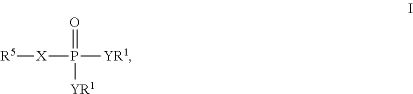

wherein X, R¹, and R⁵ are defined herein.

For example, in certain embodiments, the inhibitor is a compound of Formula II

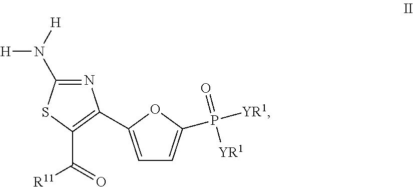

wherein R¹ and R¹¹ are defined herein. In certain embodiments, the inhibitor is

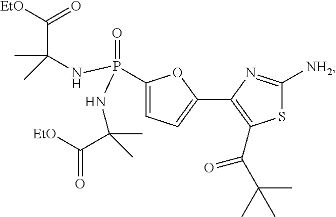 [SMILES: CCOC(=O)C(C)(C)NP(=O)(NC(C)(C)C(=O)OCC)c1ccc(-c2nc(N)sc2C(=O)C(C)(C)C)o1]

or a salt or prodrug thereof.

In other embodiments, the inhibitor is metformin.

In certain embodiments, the agent is biotin, thiamine, riboflavin, or coenzyme A, preferably biotin, thiamine, or riboflavin or a salt, ester, or prodrug thereof.

In another aspect, the invention relates to a method of treating a metabolic condition, comprising conjointly administering an inhibitor of gluconeogenesis or a salt, ester, or prodrug thereof; and at least one agent that promotes lactic acid clearance, wherein the inhibitor and the agent, taken together, are therapeutically effective.

In yet another aspect, the invention relates to a method to treat a metabolic condition, comprising administering a pharmaceutical composition disclosed herein.

In certain embodiments, the metabolic condition is diabetes, such as Type I, Type II, or gestational diabetes.

### DETAILED DESCRIPTION

Gluconeogenesis from pyruvate is a highly regulated biosynthetic pathway requiring eleven enzymes. Seven enzymes catalyze reversible reactions and are common to both gluconeogenesis and glycolysis. Four enzymes catalyze reactions unique to gluconeogenesis, namely pyruvate carboxylase, phosphoenolpyruvate carboxykinase, fructose-1,6-bisphosphatase and glucose-6-phosphatase. Overall flux through the pathway is controlled by the specific activities of these enzymes, the enzymes that catalyze the corresponding steps in the glycolytic direction, and by substrate availability. Dietary factors (glucose, fat) and hormones (e.g., insulin, glucagon, glucocorticoids, epinephrine) regulate enzyme activities in the gluconeogenesis and glycolysis pathways through gene expression and post-translational mechanisms.

Accordingly, in certain aspects, disclosed herein are compositions and methods for treating a metabolic condition comprising an inhibitor of gluconeogenesis or a salt, ester, or prodrug thereof; and at least one agent that promotes lactic acid clearance.

### I. Pharmaceutical Compositions

In one aspect, the present application provides pharmaceutical compositions comprising an inhibitor of gluconeogenesis or a salt, ester, or prodrug thereof; and at least one agent that promotes lactic acid clearance.

In certain embodiments, the inhibitor is an agent capable of interfering with gluconeogenesis. In certain embodiments, the inhibitor reduces, inhibits, or decreases the level or activity of one or more enzymes involved in gluconeogenesis. An inhibitor can be, for example, a small molecule, protein, peptide, peptidomimetic, ribozyme, nucleic acid molecule or oligonucleotide, oligosaccharide, cell, phage or virus, or a combination thereof. In certain embodiments, the inhibitor is a compound capable of inhibiting the activity of one or more of the four enzymes unique to gluconeogenesis (i.e., pyruvate carboxylase, phosphoenolpyruvate carboxykinase, fructose-1,6-bisphosphatase, and glucose-6-phosphatase). In certain embodiments, the inhibitor is a compound capable of inhibiting fructose-1,6-bisphosphatase, preferably specifically.

In certain embodiments, the inhibitor is a small molecule inhibitor of fructose-1,6-bisphosphatase. Examples of small molecule inhibitors of fructose-1,6-bisphosphatase include but are not limited to the compounds disclosed in U.S. Pat. Nos. 6,489,476 and 6,965,033 and U.S. Patent Publication No. 2007-0225259, hereby incorporated by reference in their entirety, such as the compounds of Formula I

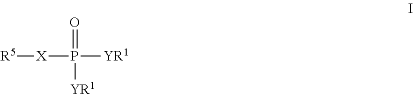

wherein R⁵ is selected from:

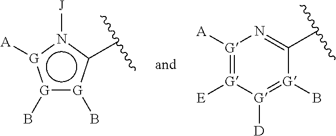

wherein:

each G is independently selected from C, N, O, S and Se, provided that no more than one G is O, S, or Se;

each G′ is independently selected from C and N such that no more than two G′ groups are N;

A is selected from —H, —NR⁴₂, —CONR⁴₂, —CO₂R³, halo, —S(O)R³, —SO₂R³, alkyl, alkenyl, alkynyl, perhaloalkyl, haloalkyl, aryl, —CH₂OH, —CH₂NR⁴₂, —CH₂CN, —CN, —C(S)NH₂, —OR³, —SR³, —N₃, —NHC(S)NR⁴₂, —NHAc, and a bond or A is absent;

each B and D are independently selected from —H, alkyl, alkenyl, alkynyl, aryl, alicyclic, aralkyl, alkoxyalkyl, —C(O)R¹¹, —C(O)SR³, —SO₂R¹¹, —S(O)R³, —CN, —NR⁹₂, —OR³, —SR³, perhaloalkyl, halo, —NO₂, and a bond, of which all except —H, —CN, perhaloalkyl, —NO₂, and halo are optionally substituted or each B and D are independently absent;

E is absent or is selected from —H, alkyl, alkenyl, alkynyl, aryl, alicyclic, alkoxyalkyl, —C(O)OR³, —CONR⁴₂, —CN, —NR⁹₂, —NO₂, —OR³, —SR³, perhaloalkyl, halo, and a bond, of which all except —H, —CN, perhaloalkyl, and halo are optionally substituted;

J is absent or is selected from —H and a bond;

X is an optionally substituted linking group that links R⁵ to the phosphorus atom via 2-4 atoms, including 0-1 heteroatoms selected from N, O, and S, except that if X is urea or carbamate there are 2 heteroatoms, measured by the shortest path between R⁵ and the phosphorus atom, and wherein the atom attached to the phosphorus is a carbon atom, and wherein there is no N in the linking group unless it is connected directly to a carbonyl or in the ring of a heterocycle; and wherein X is not a 2 carbon atom -alkyl- or -alkenyl- group; with the proviso that X is not substituted with —COOR², —SO₃R¹, or —PO₃R¹₂;

Y is independently selected from —O—, and —NR⁶—;

when Y is —O—, then R¹ attached to —O— is independently selected from —H, alkyl, optionally substituted aryl, optionally substituted alicyclic where the cyclic moiety contains a carbonate or thiocarbonate, optionally substituted -alkylaryl, —C(R²)₂OC(O)NR²₂, —NR²—C(O)—R³, —C(R²)₂—OC(O)R³, —C(R²)₂—O—C(O)OR³, —C(R²)₂OC(O)SR³, -alkyl-S—C(O)R³, -alkyl-S—S-alkylhydroxy, and -alkyl-S—S—S-alkylhydroxy,

when Y is —NR⁶—, then R¹ attached to —NR⁶— is independently selected from —H, —[C(R²)₂]_{q}—COOR³, —C(R⁴)₂COOR³, —[C(R²)₂]_{q}—C(O)SR, and -cycloalkylene-COOR³;

or when either Y is independently selected from —O— and —NR⁶—, then together R¹ and R¹ are -alkyl-S—S-alkyl- to form a cyclic group, or together R¹ and R¹ are

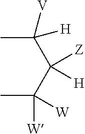

wherein

V, W, and W′ are independently selected from —H, alkyl, aralkyl, alicyclic, aryl, substituted aryl, heteroaryl, substituted heteroaryl, 1-alkenyl, and 1-alkynyl; or

together V and Z are connected via an additional 3-5 atoms to form a cyclic group containing 5-7 atoms, optionally 1 heteroatom, substituted with hydroxy, acyloxy, alkoxycarbonyloxy, or aryloxycarbonyloxy attached to a carbon atom that is three atoms from both Y groups attached to the phosphorus; or

together V and Z are connected via an additional 3-5 atoms to form a cyclic group, optionally containing 1 heteroatom, that is fused to an aryl group at the beta and gamma position to the Y attached to the phosphorus; or

together V and W are connected via an additional 3 carbon atoms to form an optionally substituted cyclic group containing 6 carbon atoms and substituted with one substituent selected from hydroxy, acyloxy, alkoxycarbonyloxy, alkylthiocarbonyloxy, and aryloxycarbonyloxy, attached to one of said carbon atoms that is three atoms from a Y attached to the phosphorus; or

together W and W′ are connected via an additional 2-5 atoms to form a cyclic group, optionally containing 0-2 heteroatoms, and V must be aryl, substituted aryl, heteroaryl, or substituted heteroaryl; and

Z is selected from —CHR²OH, —CHR²OC(O)R³, —CHR²OC(S)R³, —CHR²OC(S)OR³, —CHR²OC(O)SR³, —CHR²OCO₂R³, —OR², —SR², —CHR²N₃, —CH₂aryl, —CH(aryl)OH, —CH(CH═CR²₂)OH, —CH(C≡CR²)OH, —R², —NR²₂, —OCOR³, —OCO₂R³, —SCOR³, —SCO₂R³, —NHCOR², —NHCO₂R³, —CH₂NHaryl, —(CH₂)ₚ—OR², and —(CH₂)ₚ—SR²; or

together Z and W are connected via an additional 3-5 atoms to form a cyclic group, optionally containing one heteroatom, and V must be aryl, substituted aryl, heteroaryl, or substituted heteroaryl; or

p is an integer 2 or 3;

q is an integer 1 or 2;

with the provisos that:

a) V, Z, W, W′ are not all —H; and

b) when Z is —R², then at least one of V, W, and W′ is not —H, alkyl, aralkyl, or alicyclic;

R² is selected from R³ and —H;

R³ is selected from alkyl, aryl, alicyclic, and aralkyl;

each R⁴ is independently selected from —H, and alkyl, or together R⁴ and R⁴ form a cyclic alkyl group;

R⁶ is selected from —H, lower alkyl, acyloxyalkyl, alkoxycarbonyloxyalkyl, and lower acyl;

each R⁹ is independently selected from —H, alkyl, aralkyl, and alicyclic, or together R⁹ and R⁹ form a cyclic alkyl group;

R¹¹ is selected from alkyl (e.g., C₁-C₂₀ alkyl, C₁-C₂₀ cycloalkyl), aryl, monocyclic aryl and monocyclic heteroaryl, optionally substituted with halogen, OH, C₁-C₄ alkoxy, cyano, —NR²₂, or —OR²; and

with the provisos that:
1) when G′ is N, then the respective A, B, D, or E is absent;
2) at least one of A and B, or A, B, D, and E is not —H or is not absent;
3) when R⁵ is a six-membered ring, then X is not any 2 atom linker, an optionally substituted -alkyl-, an optionally substituted -alkenyl-, an optionally substituted -alkyloxy-, or an optionally substituted -alkylthio-;
4) when G is N, then the respective A or B is not halogen or a group directly bonded to G via a heteroatom;
5) R¹ is not unsubstituted C1-C10 alkyl;
6) when X is not an -aryl- group, then R⁵ is not substituted with two or more aryl groups;
   and pharmaceutically acceptable prodrugs and salts thereof.

In certain embodiments, R⁵ is pyrrolyl, imidazolyl, oxazolyl, thiazolyl, isothiazolyl, 1,2,4-thiadiazolyl, pyrazolyl, isoxazolyl, 1,2,3-oxadiazolyl, 1,2,4-oxadiazolyl, 1,2,5-oxadiazolyl, 1,3,4-oxadiazolyl, 1,2,4-thiadiazolyl, 1,3,4-thiadiazolyl, 1,2,3-triazolyl, 1,2,4-triazolyl, 1,2,3,4-tetrazolyl, pyridinyl, pyrimidinyl, pyrazinyl, pyridazinyl, 1,3,5-triazinyl, 1,2,4-triazinyl, or 1,3-selenazolyl, all of which contain at least one substituent.

In certain embodiments, R⁵ is selected from:

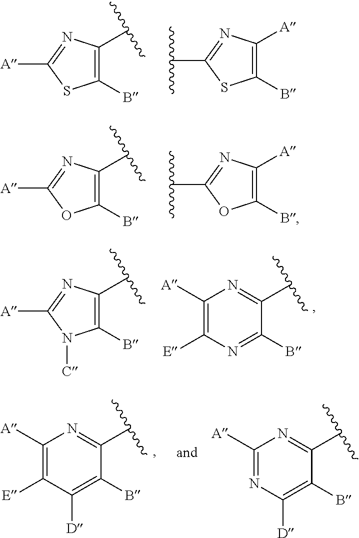

wherein

A″ is selected from —H, —NR⁴₂, —CONR⁴₂, —CO₂R³, halo, C1-C6 alkyl, C2-C6 alkenyl, C2-C6 alkynyl, C1-C6 perhaloalkyl, C1-C6 haloalkyl, aryl, —CH₂OH, —CH₂NR⁴₂, —CH₂CN, —CN, —C(S)NH₂, —OR³, —SR³, —N₃, —NHC(S)NR⁴₂, and —NHAc;

B″ and D″ are independently selected from —H, alkyl, alkenyl, alkynyl, aryl, alicyclic, aralkyl, alkoxyalkyl, —C(O)R¹¹, —C(O)SR³, —SO₂R¹¹, —S(O)R³, —CN, —NR⁹₂, —OR³, —SR³, perhaloalkyl, and halo, all except —H, —CN, perhaloalkyl, and halo are optionally substituted;

E″ is selected from —H, C1-C6 alkyl, C2-C6 alkenyl, C2-C6 alkynyl, aryl, C4-C6 alicyclic, alkoxyalkyl, —C(O)OR, —CONR⁴₂, —CN, —NR⁹₂, —OR³, C1-C6 perhaloalkyl, and halo, all except —H, —CN, perhaloalkyl, and halo are optionally substituted; and

C″ is selected from —H, alkyl, alkylalkenyl, alkylalkynyl, aryl, alicyclic, aralkyl, aryloxyalkyl, and alkoxyalkyl, all optionally substituted;

R⁴ is selected from —H and C1-C2 alkyl; and

R¹¹ is selected from alkyl, aryl, —NR²₂, and —OR².

In certain embodiments, X selected from -alkyl(hydroxy)-, -alkyl-, -alkynyl-, -aryl-, -carbonylalkyl-, -1,1-dihaloalkyl-, -alkoxyalkyl-, -alkyloxy-, -alkylthioalkyl-, -alkylthio-, -alkylaminocarbonyl-, -alkylcarbonylamino-, -alicyclic-, -aralkyl-, -alkylaryl-, -alkoxycarbonyl-, -carbonyloxyalkyl-, -alkoxycarbonyl amino-, -alkylaminocarbonylamino-, -alkylamino-, and -alkenyl-, all optionally substituted. In certain embodiments, X is selected from -heteroaryl-, -alkylcarbonylamino-, -alkylaminocarbonyl-, -alkoxycarbonyl-, and -alkoxyalkyl-.

In certain embodiments, wherein R⁵ is

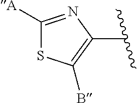

X is selected from methylenoxycarbonyl and furan-2,5-diyl; and at least one Y group is —NH—.

In certain embodiments, wherein R⁵ is

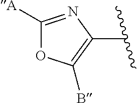

X is selected from furan-2,5-diyl and methyleneoxycarbonyl, and A″ is —NH₂; and at least one Y group is —NH—

For example, in certain embodiments, the inhibitor is a compound of formula II

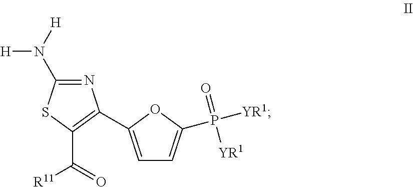

preferably

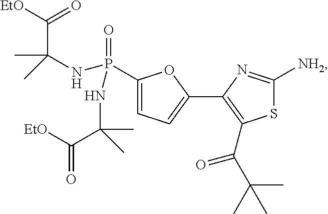 [SMILES: CCOC(=O)C(C)(C)NP(=O)(NC(C)(C)C(=O)OCC)c1ccc(-c2nc(N)sc2C(=O)C(C)(C)C)o1]

or a salt or prodrug thereof. In other embodiments, the inhibitor is metformin.

Exemplary inhibitors include those highlighted in Table 1.

TABLE 1
Exemplary Inhibitors

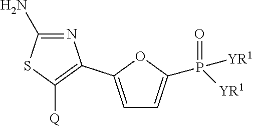

| Compound | | |
|---|---|---|
| # | Q | YR¹ |
| 1.1 | 2,2-dimethylpropionyl | —OH |
| 1.2 | 2,2-dimethylbutyryl | —OH |
| 1.3 | 2-Ethyl-2-Methylbutyryl | —OH |
| 1.4 | Acetyl | —OH |
| 1.5 | Benzoyl | —OH |
| 2.1 | 2,2-Dimethylpropionyl | —NHC(Me)₂CO₂Et |
| 2.2 | 2,2-Dimethylpropionyl | —NHCH(Me)CO₂Et (S) |
| 2.3 | 2,2-Dimethylpropionyl | —NHC(Me)₂CO₂i-Pr |
| 2.4 | 2,2-Dimethylpropionyl | —NHCH₂CO₂Et |
| 2.5 | 2-Ethyl-2-Methylbutyryl | —NHC(Me)₂CO₂Et |
| 2.6 | 2-Ethyl-2-Methylbutyryl | —NHC(Me)₂CO₂i-Pr |
| 2.7 | 2-Ethyl-2-Methylbutyryl | —NHCH(Me)CO₂Et (S) |
| 2.8 | 2,2-Dimethylbutyryl | —NHC(Me)₂CO₂i-Pr |
| 2.9 | 2,2-Dimethylbutyryl | —NHC(Me)₂CO₂Et |
| 2.10 | 2,2-Dimethylbutyryl | —NHCH(Me)CO₂Et (S) |
| 2.11 | 2-Ethyl-2-Methylbutyryl | —NHCH(Me)CO₂i-Pr (S) |
| 2.12 | 2,2-Dimethylbutyryl | —NHCH₂CO₂t-Bu |
| 2.13 | 2,2-Dimethylpropionyl | —NHCH(Me)CO₂i-Pr (S) |
| 3.1 | 2,2-Dimethylpropyl | —OH |
| 3.2 | Cyclopentylmethyl | —OH |
| 3.3 | 2,2-Dimethylbutyl | —OH |
| 3.4 | 2-Propyl | —OH |
| 3.5 | 2-Methylbutyl | —OH |
| 3.6 | 2-Methylpropyl | —OH |
| 4.1 | 2,2-Dimethylpropyl | —NHCH(Me)CO₂Et (S) |
| 4.2 | Phenyl | —NHCH(Me)CO₂Et (S) |
| 4.3 | Cyclohexyl | —NHCH(Me)CO₂i-Pr (S) |
| 4.4 | 2,2-Dimethylpropyl | —NHCH₂CO₂Et |
| 4.5 | 2,2,3-Trimethylbutyl | —NHCH(Me)CO₂Et (S) |
| 4.6 | 2-Methylpropyl | —NHCH(Me)CO₂Et (S) |

The pharmaceutical compositions disclosed herein also comprise an agent that promotes lactic acid clearance. In certain embodiments, the agent that promotes lactic acid clearance is an agent that accelerates the elimination of lactic acid from a subject and/or reduces the endogenous production of lactic acid in a subject. In certain embodiments, the agent may be a coenzyme or cofactor such as biotin, thiamine, riboflavin, or coenzyme A, preferably biotin, thiamine, or riboflavin. In certain embodiments, the agent may be a prodrug or derivative of an agent that is converted to biotin, thiamine, riboflavin, or coenzyme A, preferably biotin, thiamine, or riboflavin.

The pharmaceutical compositions disclosed herein are useful in treating a metabolic condition, such as hyperglycaemia, diabetes, eating disorders, and/or obesity. In certain embodiments, the metabolic condition is diabetes (e.g., Type I, Type II, and gestational diabetes).

This invention includes the use of pharmaceutically acceptable salts of inhibitors and agents of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, pharmaceutically acceptable salts include salts of inhibitors or agents and their prodrugs derived from the combination of an inhibitor or agent and an organic or inorganic acid or base. Suitable acids include acetic acid, adipic acid, benzenesulfonic acid, (+)-7,7-dimethyl-2-oxobicyclo[2.2.1]heptane-1-methane sulfonic acid, citric acid, 1,2-ethanedisulfonic acid, dodecyl sulfonic acid, fumaric acid, glucoheptonic acid, gluconic acid, glucuronic acid, hippuric acid, HBr, HCl, HI, 2-hydroxyethanesulfonic acid, lactic acid, lactobionic acid, maleic acid, methane sulfonic acid, methylbromide acid, methyl sulfuric acid, 2-naphthalenesulfonic acid, nitric acid, oleic acid, 4,4′ methylenebis[3-hydroxy-2-naphthalenecarboxylic acid], phosphoric acid, polygalacturonic acid, stearic acid, succinic acid, sulfuric acid, sulfosalicylic acid, tannic acid, tartaric acid, terphthalic acid, and p-toluenesulfonic acid.

The pharmaceutically acceptable salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

The compositions and methods of the present invention may be utilized to treat an individual in need thereof. In certain embodiments, the individual is a mammal such as a human, or a non-human mammal. When administered to an animal, such as a human, the composition is preferably administered as a pharmaceutical composition comprising, for example, an inhibitor and agent combination of the invention and a pharmaceutically acceptable carrier. For example, the different therapeutic compounds (e.g., an inhibitor of gluconeogenesis, an agent that promotes lactic acid clearance, etc.) can be administered either in the same formulation or in a separate formulation, either concomitantly or sequentially. Oral routes of administration are preferred. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as an inhibitor and agent combination of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, an inhibitor and/or agent combination of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated and/or the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as an inhibitor and agent combination of the invention, with the carrier and, optionally, one or more accessory ingredients. In general, the formulations are prepared by uniformly and intimately bringing into association an inhibitor and agent combination of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an inhibitor and agent combination of the present invention as active ingredients. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for administration to the mouth may be presented as a mouthwash, or an oral spray, or an oral ointment.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations which are suitable for vaginal administration also include pessaries, tampons, creams, gels, pastes, foams or spray formulations containing such carriers as are known in the art to be appropriate.

Alternatively or additionally, compositions can be formulated for delivery via a catheter, stent, wire, or other intraluminal device. Delivery via such devices may be especially useful for delivery to the bladder, urethra, ureter, rectum, or intestine.

Dosage forms for the topical administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the inhibitor and agent combination of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above. In certain embodiments, a suitable daily dose is between about 0.01 mg and 2500 mg. For example, a suitable daily dose is 10, 50, 100, 150, 200, 300, 400, 500, 750, or 1000 mg of the inhibitor. The dose may be administered in as many divided doses as is convenient.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment may be any animal in need, including primates, in particular humans, and other mammals such as equines, cattle, swine and sheep; and poultry and pets in general.

In certain embodiments, compositions of the invention may be used alone or conjointly administered with another therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered simultaneously, or within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration with one or more additional therapeutic agent(s) (e.g., one or more additional anti-diabetic agent(s)) provides improved efficacy relative to each individual administration of the inhibitor and agent combination of the invention or the one or more additional therapeutic agent(s). In certain such embodiments, the conjoint administration provides an additive effect, wherein an additive effect refers to the sum of each of the effects of individual administration of the inhibitor and agent combination of the invention and the one or more additional therapeutic agent(s). For

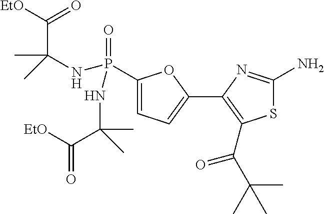 [SMILES: CCOC(=O)C(C)(C)NP(=O)(NC(C)(C)C(=O)OCC)c1ccc(-c2nc(N)sc2C(=O)C(C)(C)C)o1]

example, if the composition comprises as the inhibitor, it may be administered conjointly with metformin. Similarly, if the composition comprises metformin, it may be administered conjointly with

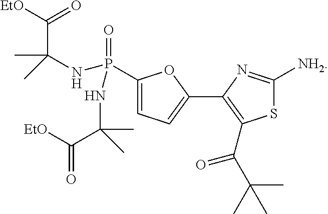 [SMILES: CCOC(=O)C(C)(C)NP(=O)(NC(C)(C)C(=O)OCC)c1ccc(-c2nc(N)sc2C(=O)C(C)(C)C)o1]

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

An effective amount of the composition may be administered in a single dose per day or in fractional doses over the day, for example two to three times a day. By way of example, the administration of a composition according to the invention may be performed at a rate, for example, of 3 times a day or more, generally over a prolonged period of at least a week, 2 weeks, 3 weeks, 4 weeks, or even 4 to 15 weeks, optionally comprising one or more periods of stoppage or being repeated after a period of stoppage.

As one of skill in the art will appreciate, compositions of the present invention, not having adverse effects upon administration to a subject, may be administered daily to the subject.

Preferred embodiments of this invention are described herein. Of course, variations, changes, modifications and substitution of equivalents of those preferred embodiments will become apparent to those of ordinary skill in the art upon reading the foregoing description. The inventors expect skilled artisans to employ such variations, changes, modifications and substitution of equivalents as appropriate, and the inventors intend for the invention to be practiced otherwise than specifically described herein. Those of skill in the art will readily recognize a variety of non-critical parameters that could be changed, altered or modified to yield essentially similar results. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### II. Methods of Use

In certain aspects, provided herein are methods of treating a metabolic condition, comprising conjointly administering an inhibitor of gluconeogenesis or a salt, ester, or prodrug thereof; and at least one agent that promotes lactic acid clearance. Suitable inhibitors and agents are disclosed throughout this application.

In some embodiments, the inhibitor of gluconeogenesis and the agent that promotes lactic acid clearance are co-formulated. For example, in some embodiments, provided herein are methods to treat a metabolic condition comprising administering a pharmaceutical composition disclosed herein.

In some embodiments, the metabolic condition is diabetes (e.g., Type I, Type II, or gestational diabetes).

### III. Definitions

For purposes of the present invention, the following definitions will be used (unless expressly stated otherwise):

X group nomenclature as used herein in Formula I describes the group attached to the phosphonate and ends with the group attached to the heteroaromatic ring. For example, when X is alkylamino, the following structure is intended:

(heteroaromatic ring)-NR-alk-P(O)(OR¹)₂

Likewise, A, B, C, D, E, A″, B″, C″, D″ and E″ groups and other substituents of the heteroaromatic ring are described in such a way that the term ends with the group attached to the heteroaromatic ring. Generally, substituents are named such that the term ends with the group at the point of attachment.

The term “aryl” refers to aromatic groups which have 5-14 ring atoms and at least one ring having a conjugated pi electron system and includes carbocyclic aryl, heterocyclic aryl and biaryl groups, all of which may be optionally substituted. Suitable aryl groups include phenyl and furan-2,5-diyl.

Carbocyclic aryl groups are groups wherein the ring atoms on the aromatic ring are carbon atoms. Carbocyclic aryl groups include monocyclic carbocyclic aryl groups and polycyclic or fused compounds such as optionally substituted naphthyl groups.

Heterocyclic aryl or heteroaryl groups are groups having from 1 to 4 heteroatoms as ring atoms in the aromatic ring and the remainder of the ring atoms being carbon atoms. Suitable heteroatoms include oxygen, sulfur, nitrogen, and selenium. Suitable heteroaryl groups include furanyl, thienyl, pyridyl, pyrrolyl, N-lower alkyl pyrrolyl, pyridyl-N-oxide, pyrimidyl, pyrazinyl, imidazolyl, and the like, all optionally substituted.

The term “annulation” or “annulated” refers to the formation of an additional cyclic moiety onto an existing aryl or heteroaryl group. The newly formed ring may be carbocyclic or heterocyclic, saturated or unsaturated, and contains 2-9 new atoms of which 0-3 may be heteroatoms taken from the group of N, O, and S. The annulation may incorporate atoms from the X group as part of the newly formed ring.

The term “biaryl” represents aryl groups containing more than one aromatic ring including both fused ring systems and aryl groups substituted with other aryl groups. Such groups may be optionally substituted. Suitable biaryl groups include naphthyl and biphenyl.

The term “alicyclic” means compounds which combine the properties of aliphatic and cyclic compounds. Such cyclic compounds include but are not limited to, aromatic, cycloalkyl and bridged cycloalkyl compounds. The cyclic compound includes heterocycles. Cyclohexenylethyl and cyclohexylethyl are suitable alicyclic groups. Such groups may be optionally substituted.

The term “optionally substituted” or “substituted” includes groups substituted by one to four substituents, independently selected from lower alkyl, lower aryl, lower aralkyl, lower alicyclic, hydroxy, lower alkoxy, lower aryloxy, perhaloalkoxy, aralkoxy, heteroaryl, heteroaryloxy, heteroarylalkyl, heteroaralkoxy, azido, amino, guanidino, amidino, halo, lower alkylthio, oxo, acylalkyl, carboxy esters, carboxyl, carboxamido, nitro, acyloxy, aminoalkyl, alkylaminoaryl, alkylaryl, alkylaminoalkyl, alkoxyaryl, arylamino, aralkylamino, phosphono, sulfonyl, carboxamidoalkylaryl, carboxamidoaryl, hydroxyalkyl, haloalkyl, alkylaminoalkylcarboxy-, aminocarboxamidoalkyl-, cyano, lower alkoxyalkyl, lower perhaloalkyl, and arylalkyloxyalkyl. “Substituted aryl” and “substituted heteroaryl” preferably refers to aryl and heteroaryl groups substituted with 1-3 substituents. Preferably these substituents are selected from lower alkyl, lower alkoxy, lower perhaloalkyl, halo, hydroxy, and amino. “Substituted” when describing an R⁵ group does not include annulation.

The term “aralkyl” refers to an alkyl group substituted with an aryl group. Suitable aralkyl groups include benzyl, picolyl, and the like, and may be optionally substituted. The term “-aralkyl-” refers to a divalent group -aryl-alkylene-. “Heteroarylalkyl” refers to an alkylene group substituted with a heteroaryl group.

The term “-alkylaryl-” refers to the group -alk-aryl- where “alk” is an alkylene group. “Lower -alkylaryl-” refers to such groups where alkylene is lower alkylene.

The term “lower” referred to herein in connection with organic radicals or compounds respectively defines such as with up to and including 10, preferably up to and including 6, and advantageously one to four carbon atoms. Such groups may be straight chain, branched, or cyclic.

The terms “arylamino” (a), and “aralkylamino” (b), respectively, refer to the group —NRR′ wherein respectively, (a) R is aryl and R′ is hydrogen, alkyl, aralkyl or aryl, and (b) R is aralkyl and R′ is hydrogen or aralkyl, aryl, alkyl.

The term “acyl” refers to —C(O)R where R is alkyl and aryl.

The term “carboxy esters” refers to —C(O)OR where R is alkyl, aryl, aralkyl, and alicyclic, all optionally substituted.

The term “carboxyl” refers to —C(O)OH.

The term “oxo” refers to ═O in an alkyl group.

The term “amino” refers to —NRR′ where R and R′ are independently selected from hydrogen, alkyl, aryl, aralkyl and alicyclic, all except H are optionally substituted; and R and R′ can form a cyclic ring system.

The term “carbonylamino” and “-carbonylamino-” refers to RCONR— and —CONR—, respectively, where each R is independently hydrogen or alkyl.

The term “halogen” or “halo” refers to —F, —Cl, —Br and —I.

The term “-oxyalkylamino-” refers to —O-alk-NR—, where “alk” is an alkylene group and R is H or alkyl.

The term “-alkylaminoalkylcarboxy-” refers to the group -alk-NR-alk-C(O)—O— where “alk” is an alkylene group, and R is a H or lower alkyl.

The term “-alkylaminocarbonyl-” refers to the group -alk-NR—C(O)— where “alk” is an alkylene group, and R is a H or lower alkyl.

The term “-oxyalkyl-” refers to the group —O-alk- where “alk” is an alkylene group.

The term “-alkylcarboxyalkyl-” refers to the group -alk-C(O)—O-alk- where each alk is independently an alkylene group.

The term “alkyl” refers to saturated aliphatic groups including straight-chain, branched chain and cyclic groups. Alkyl groups may be optionally substituted. Suitable alkyl groups include methyl, isopropyl, and cyclopropyl.

The term “cyclic alkyl” or “cycloalkyl” refers to alkyl groups that are cyclic. Suitable cyclic groups include norbornyl and cyclopropyl. Such groups may be substituted.

The term “heterocyclic” and “heterocyclic alkyl” refer to cyclic groups of 3 to 10 atoms, more preferably 3 to 6 atoms, containing at least one heteroatom, preferably 1 to 3 heteroaroms. Suitable heteroatoms include oxygen, sulfur, and nitrogen. Heterocyclic groups may be attached through a nitrogen or through a carbon atom in the ring. Suitable heterocyclic groups include pyrrolidinyl, morpholino, morpholinoethyl, and pyridyl.

The term “phosphono” refers to —PO₃R₂, where each R is independently selected from —H, alkyl, aryl, aralkyl, and alicyclic.

The term “sulphonyl” or “sulfonyl” refers to —SO₃R, where R is H, alkyl, aryl, aralkyl, and alicyclic.

The term “alkenyl” refers to unsaturated groups which contain at least one carbon-carbon double bond and includes straight-chain, branched-chain and cyclic groups. Alkenyl groups may be optionally substituted. Suitable alkenyl groups include allyl. “1-alkenyl” refers to alkenyl groups where the double bond is between the first and second carbon atom. If the 1-alkenyl group is attached to another group, e.g., it is a W substituent attached to the cyclic phosph(oramid)ate, it is attached at the first carbon.

The term “alkynyl” refers to unsaturated groups which contain at least one carbon-carbon triple bond and includes straight-chain, branched-chain and cyclic groups. Alkynyl groups may be optionally substituted. Suitable alkynyl groups include ethynyl. “1-alkynyl” refers to alkynyl groups where the triple bond is between the first and second carbon atom. If the 1-alkynyl group is attached to another group, e.g., it is a W substituent attached to the cyclic phosph(oramid)ate, it is attached at the first carbon.

The term “alkylene” refers to a divalent straight chain, branched chain or cyclic saturated aliphatic group.

The term “-cycloalkylene-COOR³” refers to a divalent cyclic alkyl group or heterocyclic group containing 4 to 6 atoms in the ring, with 0-1 heteroatoms selected from 0, N, and S. The cyclic alkyl or heterocyclic group is substituted with —COOR³.

The term “acyloxy” refers to the ester group —O—C(O)R, where R is H, alkyl, alkenyl, alkynyl, aryl, aralkyl, or alicyclic.

The term “aminoalkyl-” refers to the group NR₂-alk- wherein “alk” is an alkylene group and each R is independently selected from H, alkyl, aryl, aralkyl, and alicyclic.

The term “-alkyl(hydroxy)-” refers to an —OH off the alkyl chain. When this term is an X group, the —OH is at the position a to the phosphorus atom.

The term “alkylaminoalkyl-” refers to the group alkyl-NR-alk- wherein each “alk” is an independently selected alkylene, and R is H or lower alkyl. “Lower alkylaminoalkyl-” refers to groups where each alkylene group is lower alkylene.

The term “arylaminoalkyl-” refers to the group aryl-NR-alk- wherein “alk” is an alkylene group and R is H, alkyl, aryl, aralkyl, and alicyclic. In “lower arylaminoalkyl-”, the alkylene group is lower alkylene.

The term “alkylaminoaryl-” refers to the group alkyl-NR-aryl- wherein “aryl” is a divalent group and R is H, alkyl, aralkyl, and alicyclic. In “lower alkylaminoaryl-”, the alkylene group is lower alkyl.

The term “alkyloxyaryl-” refers to an aryl group substituted with an alkyloxy group. In “lower alkyloxyaryl-”, the alkyl group is lower alkyl.

The term “aryloxyalkyl-” refers to an alkyl group substituted with an aryloxy group.

The term “aralkyloxyalkyl-” refers to the group aryl-alk-O-alk- wherein “alk” is an alkylene group. “Lower aralkyloxyalkyl-” refers to such groups where the alkylene groups are lower alkylene.

The term “-alkoxy-” or “-alkyloxy-” refers to the group -alk-O— wherein “alk” is an alkylene group. The term “alkoxy-” refers to the group alkyl-O—.

The term “-alkoxyalkyl-” or “-alkyloxyalkyl-” refer to the group -alk-O-alk- wherein each “alk” is an independently selected alkylene group. In “lower -alkoxyalkyl-”, each alkylene is lower alkylene.

The terms “alkylthio-” and “-alkylthio-” refer to the groups alkyl-S—, and -alk-S—, respectively, wherein “alk” is alkylene group.

The term “-alkylthioalkyl-” refers to the group -alk-S-alk- wherein each “alk” is an independently selected alkylene group. In “lower -alkylthioalkyl-” each alkylene is lower alkylene.

The term “alkoxycarbonyloxy-” refers to alkyl-O—C(O)—O—.

The term “aryloxycarbonyloxy-” refers to aryl-O—C(O)—O—.

The term “alkylthiocarbonyloxy-” refers to alkyl-S—C(O)—O—.

The term “-alkoxycarbonylamino-” refers to -alk-O—C(O)—NR¹—, where “alk” is alkylene and R¹ includes —H, alkyl, aryl, alicyclic, and aralkyl.

The term “-alkylaminocarbonylamino-” refers to -alk-NR¹—C(O)—NR¹—, where “alk” is alkylene and R¹ is independently selected from H, alkyl, aryl, aralkyl, and alicyclic.

The terms “amido” or “carboxamido” refer to NR₂—C(O)— and RC(O)—NR¹—, where each R and R¹ is independently selected from H, alkyl, aryl, aralkyl, and alicyclic. The term does not include urea, —NR—C(O)—NR—.

The terms “carboxamidoalkylaryl” and “carboxamidoaryl” refers to an aryl-alk-NR¹—C(O)—, and an —NR¹—C(O)-alk-, respectively, where “ar” is aryl, and “alk” is alkylene, R¹ and R include H, alkyl, aryl, aralkyl, and aliyclic.

The term “-alkylcarboxamido-” or “-alkylcarbonylamino-” refers to the group -alk-C(O)N(R)— wherein “alk” is an alkylene group and R is H or lower alkyl.

The term “-alkylaminocarbonyl-” refers to the group -alk-NR—C(O)— wherein “alk” is an alkylene group and R is H or lower alkyl.

The term “aminocarboxamidoalkyl-” refers to the group NR₂—C(O)—N(R)-alk- wherein each R independently is an alkyl group or H and “alk” is an alkylene group. “Lower aminocarboxamidoalkyl-” refers to such groups wherein “alk” is lower alkylene.

The term “thiocarbonate” refers to —O—C(S)—O— either in a chain or in a cyclic group.

The term “hydroxyalkyl” refers to an alkyl group substituted with one —OH.

The term “haloalkyl” refers to an alkyl group substituted with one halo, selected from the group I, Cl, Br, F.

The term “cyano” refers to —C≡N.

The term “nitro” refers to —NO₂.

The term “acylalkyl” refers to an alkyl-C(O)-alk-, where “alk” is alkylene.

The term “heteroarylalkyl” refers to an alkyl group substituted with a heteroaryl group.

The term “-1,1-dihaloalkyl-” refers to an X group where the 1 position and therefore halogens are a to the phosphorus atom.

The term “perhalo” refers to groups wherein every C—H bond has been replaced with a C-halo bond on an aliphatic or aryl group. Suitable perhaloalkyl groups include —CF₃ and —CFCl₂.

The term “guanidino” refers to both —NR—C(NR)—NR₂ as well as —N═C(NR₂)₂ where each R group is independently selected from the group of —H, alkyl, alkenyl, alkynyl, aryl, and alicyclic, of which all except —H are optionally substituted.

The term “amidino” refers to —C(NR)—NR₂ where each R group is independently selected from the group of —H, alkyl, alkenyl, alkynyl, aryl, and alicyclic, of which all except —H are optionally substituted.

The term “cyclic 1′,3′-propane ester”, “cyclic 1,3-propane ester”, “cyclic 1′,3′-propanyl ester”, and “cyclic 1,3-propanyl ester” refers to the following:

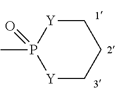

The phrase “together V and Z are connected via an additional 3-5 atoms to form a cyclic group containing 5-7 atoms, optionally containing 1 heteroatom, substituted with hydroxy, acyloxy, alkoxycarbonyloxy, or aryloxycarbonyloxy attached to a carbon atom that is three atoms from both Y groups attached to the phosphorus” includes, for example, the following:

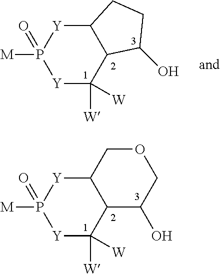

The structure shown above (left) has an additional 3 carbon atoms that forms a five-membered cyclic group. Such cyclic groups must possess the listed substitution to be oxidized.

The phrase “together V and Z are connected via an additional 3-5 atoms to form a cyclic group, optionally containing one heteroatom, that is fused to an aryl group attached at the beta and gamma position to the Y attached to the phosphorus” includes, for example, the following:

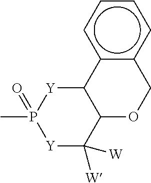

The phrase “together V and W are connected via an additional 3 carbon atoms to form an optionally substituted cyclic group containing 6 carbon atoms and substituted with one substituent selected from hydroxy, acyloxy, alkoxycarbonyloxy, alkylthiocarbonyloxy, and aryloxycarbonyloxy attached to one of said additional carbon atoms that is three atoms from a Y attached to the phosphorus” includes, for example, the following:

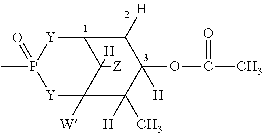

The structure above has an acyloxy substituent that is three carbon atoms from a Y, and an optional substituent, —CH₃, on the new 6-membered ring. There has to be at least one hydrogen at each of the following positions: the carbon attached to Z; both carbons alpha to the carbon labelled “3”; and the carbon attached to “OC(O)CH₃” above.

The phrase “together W and W′ are connected via an additional 2-5 atoms to form a cyclic group, optionally containing 0-2 heteroatoms, and V must be aryl, substituted aryl, heteroaryl, or substituted heteroaryl” includes, for example, the following:

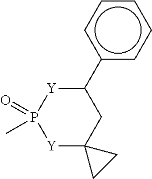

The structure above has V=aryl, and a spiro-fused cyclopropyl group for W and W′.

The term “cyclic phosph(oramid)ate” refers to

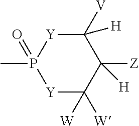

where Y is independently —O— or —NR⁶—. The carbon attached to V must have a C—H bond. The carbon attached to Z must also have a C—H bond.

As used herein, the term “administering” means the actual physical introduction of a composition into or onto (as appropriate) a subject. Any and all methods of introducing the composition into subject are contemplated according to the invention; the method is not dependent on any particular means of introduction and is not to be so construed. Means of introduction are well-known to those skilled in the art, and also are exemplified herein.

As used herein, the terms “effective amount”, “effective dose”, “sufficient amount”, “amount effective to”, “therapeutically effective amount” or grammatical equivalents thereof mean a dosage sufficient to produce a desired result, to ameliorate, or in some manner, reduce a symptom or stop or reverse progression of a condition and provide either a subjective relief of a symptom(s) or an objectively identifiable improvement as noted by a clinician or other qualified observer. Amelioration of a symptom of a particular condition by administration of a pharmaceutical composition described herein refers to any lessening, whether permanent or temporary, lasting, or transitory, that can be associated with the administration of the pharmaceutical composition.

As used herein, the term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds in a formulation represented above can be replaced with the corresponding suitable prodrug, e.g., wherein a hydroxyl in the parent compound is presented as an ester or a carbonate or carboxylic acid present in the parent compound is presented as an ester.

Standard prodrugs are formed using groups attached to functionality, e.g., HO—, HS—, HOOC—, R₂N—, associated with the FBPase inhibitor, that cleave in vivo. Standard prodrugs include but are not limited to carboxylate esters where the group is alkyl, aryl, aralkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Standard prodrugs of phosphonic acids are also included and may be represented by R¹ in formula I. The groups illustrated are exemplary, not exhaustive, and one skilled in the art could prepare other known varieties of prodrugs. Such prodrugs of the compounds of formula I fall within the scope of the present invention. Prodrugs must undergo some form of a chemical transformation to produce the compound that is biologically active or is a precursor of the biologically active compound. In some cases, the prodrug is biologically active usually less than the drug itself, and serves to improve efficacy or safety through improved oral bioavailability, pharmacodynamic half-life, etc.

The term “prodrug ester” as employed herein includes, but is not limited to, the following groups and combinations of these groups:

[1] Acyloxyalkyl esters which are well described in the literature (Farquhar et al., J. Pharm. Sci. 72, 324-325 (1983)) and are represented by formula A

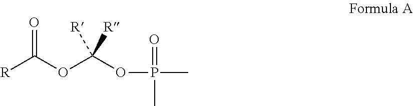

wherein R, R′, and R″ are independently H, alkyl, aryl, alkylaryl, and alicyclic; (see WO 90/08155; WO 90/10636).

[2] Other acyloxyalkyl esters are possible in which an alicyclic ring is formed, such as is shown in formula B. These esters have been shown to generate phosphorus-containing nucleotides inside cells through a postulated sequence of reactions beginning with deesterification and followed by a series of elimination reactions (e.g., Freed et al., Biochem. Pharm. 38: 3193-3198 (1989)).

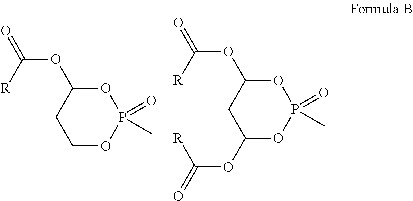

wherein R is —H, alkyl, aryl, alkylaryl, alkoxy, aryloxy, alkylthio, arylthio, alkylamino, arylamino, cycloalkyl, or alicyclic.

[3] Another class of these double esters known as alkyloxycarbonyloxymethyl esters, as shown in formula A, where R is alkoxy, aryloxy, alkylthio, arylthio, alkylamino, and arylamino; R′, and R″ are independently H, alkyl, aryl, alkylaryl, and alicyclic, have been studied in the area of β-lactam antibiotics (Tatsuo Nishimura et al. J. Antibiotics, 1987, 40(1), 81-90; for a review see Ferres, H., Drugs of Today, 1983, 19, 499.). More recently Cathy, M. S., et al. (Abstract from AAPS Western Regional Meeting, April, 1997) showed that these alkyloxycarbonyloxymethyl ester prodrugs on (9-[(R)-2-phosphonomethoxy)propyl]adenine (PMPA) are bioavailable up to 30% in dogs.

[4] Aryl esters have also been used as phosphonate prodrugs (e.g. Erion, DeLambert et al., J. Med. Chem. 37: 498, 1994; Serafinowska et al., J. Med. Chem. 38: 1372, 1995). Phenyl as well as mono and poly-substituted phenyl proesters have generated the parent phosphonic acid in studies conducted in animals and in man (Formula C). Another approach has been described where Y is a carboxylic ester ortho to the phosphate. Khamnei and Torrence, J. Med. Chem.; 39:4109-4115 (1996).

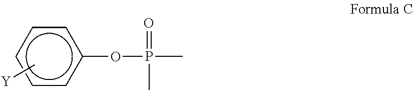

wherein Y is H, alkyl, aryl, alkylaryl, alkoxy, acyloxy, halogen, amino, alkoxycarbonyl, hydroxy, cyano, and alicyclic.

[5] Benzyl esters have also been reported to generate the parent phosphonic acid. In some cases, using substituents at the para-position can accelerate the hydrolysis. Benzyl analogs with 4-acyloxy or 4-alkyloxy group [Formula D, X═H, OR or O(CO)R or O(CO)OR] can generate the 4-hydroxy compound more readily through the action of enzymes, e.g. oxidases, esterases, etc. Examples of this class of prodrugs are described in Mitchell et al., J. Chem. Soc. Perkin Trans. I 2345 (1992); Brook, et al. WO 91/19721.

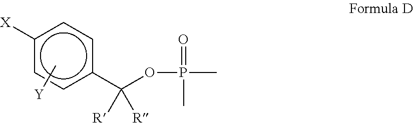

wherein X and Y are independently H, alkyl, aryl, alkylaryl, alkoxy, acyloxy, hydroxy, cyano, nitro, perhaloalkyl, halo, or alkyloxycarbonyl; and

R′ and R″ are independently H, alkyl, aryl, alkylaryl, halogen, and alicyclic.

[6] Thio-containing phosphonate proesters have been described that are useful in the delivery of FBPase inhibitors to hepatocytes. These proesters contain a protected thioethyl moiety as shown in formula E. One or more of the oxygens of the phosphonate can be esterified. Since the mechanism that results in de-esterification requires the generation of a free thiolate, a variety of thiol protecting groups are possible. For example, the disulfide is reduced by a reductase-mediated process (Puech et al., Antiviral Res., 22: 155-174 (1993)). Thioesters will also generate free thiolates after esterase-mediated hydrolysis. Benzaria, et al., J. Med. Chem., 39:4958 (1996). Cyclic analogs are also possible and were shown to liberate phosphonate in isolated rat hepatocytes.

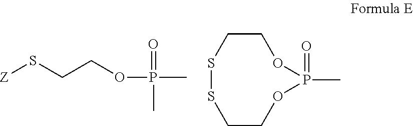

wherein Z is alkylcarbonyl, alkoxycarbonyl, arylcarbonyl, aryloxycarbonyl, or alkylthio.

Other examples of suitable prodrugs include proester classes exemplified by Biller and Magnin (U.S. Pat. No. 5,157,027); Serafinowska et al. (J. Med. Chem. 38, 1372 (1995)); Starrett et al. (J. Med. Chem. 37, 1857 (1994)); Martin et al. J. Pharm. Sci. 76, 180 (1987); Alexander et al., Collect. Czech. Chem. Commun, 59, 1853 (1994)); and EPO patent application 0 632 048 A1. Some of the structural classes described are optionally substituted, including fused lactones attached at the omega position (formulae E-1 and E-2) and optionally substituted 2-oxo-1,3-dioxolenes attached through a methylene to the phosphorus oxygen (formula E-3) such as:

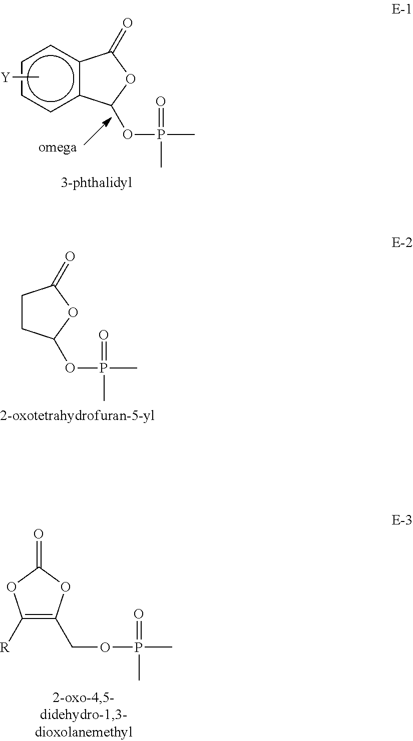

wherein R is —H, alkyl, cycloalkyl, or alicyclic; and

wherein Y is —H, alkyl, aryl, alkylaryl, cyano, alkoxy, acyloxy, halogen, amino, alicyclic, and alkoxycarbonyl.

The prodrugs of Formula E-3 are an example of “optionally substituted alicyclic where the cyclic moiety contains a carbonate or thiocarbonate.”

[7] Propyl phosphonate proesters can also be used to deliver FBPase inhibitors into hepatocytes. These proesters may contain a hydroxyl and hydroxyl group derivatives at the 3-position of the propyl group as shown in formula F. The R and X groups can form a cyclic ring system as shown in formula F. One or more of the oxygens of the phosphonate can be esterified.

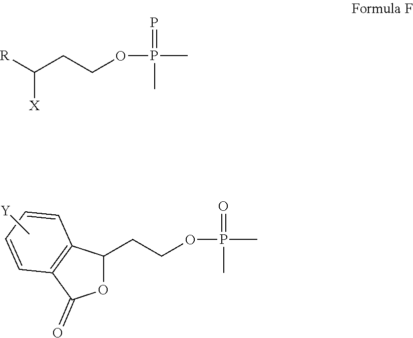

wherein R is alkyl, aryl, heteroaryl;
X is hydrogen, alkylcarbonyloxy, alkyloxycarbonyloxy; and
Y is alkyl, aryl, heteroaryl, alkoxy, alkylamino, alkylthio, halogen, hydrogen, hydroxy, acyloxy, amino.

[8] Phosphoramidate derivatives have been explored as phosphate prodrugs (e.g., McGuigan et al., J. Med. Chem., 1999, 42: 393 and references cited therein) as shown in Formula G and H.

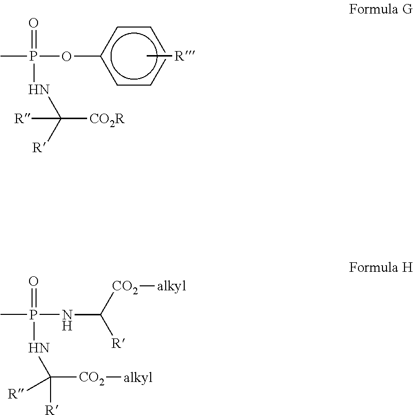

Cyclic phosphoramidates have also been studied as phosphonate prodrugs because of their speculated higher stability compared to non-cyclic phosphoramidates (e.g. Starrett et al., J. Med. Chem., 1994, 37: 1857.

Another type of nucleotide prodrug was reported as the combination of S-acyl-2-thioethyl ester and phosphoramidate (Egron et al., Nucleosides & Nucleotides, 1999, 18, 981) as shown in Formula K.

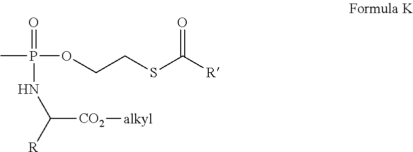

Other prodrugs are possible based on literature reports such as substituted ethyls for example, bis(trichloroethyl)esters as disclosed by McGuigan, et al. Bioorg Med. Chem. Lett., 3:1207-1210 (1993), and the phenyl and benzyl combined nucleotide esters reported by Meier, C. et al. Bioorg. Med. Chem. Lett., 7:99-104 (1997).

The structure

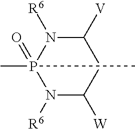

has a plane of symmetry running through the phosphorus-oxygen double bond when R⁶═R⁶, V═W, W′═H, and V and W are either both pointing up or both pointing down. The same is true of structures where each —NR⁶ is replaced with —O—.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

As used herein, a “subject” means a human or animal (in the case of an animal, more typically a mammal). In one aspect, the subject is a human.

The term “treating” is art-recognized and includes administration to the host of one or more of the subject compositions, e.g., to diminish, ameliorate, or stabilize the existing unwanted condition or side effects thereof.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A pharmaceutical composition comprising an inhibitor of gluconeogenesis or a salt, ester, or prodrug thereof; and at least one agent that promotes lactic acid clearance.

2. The pharmaceutical composition of claim 1, wherein the inhibitor is an inhibitor of fructose-1,6-bisphosphatase.

3. The pharmaceutical composition of claim 1, wherein the inhibitor is a compound of formula I 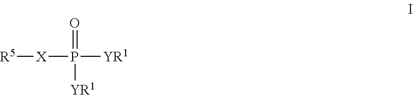
wherein R⁵ is selected from: 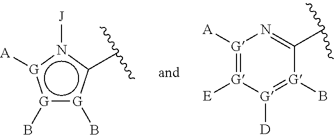
wherein:
each G is independently selected from C, N, O, S and Se, and wherein only one G may be O, S, or Se;
each G′ is independently selected from C and N and wherein no more than two G′ groups are N;
A is selected from —H, —NR⁴₂, —CONR⁴₂, —CO₂R³, halo, —S(O)R³, —SO₂R³, alkyl, alkenyl, alkynyl, perhaloalkyl, haloalkyl, aryl, —CH₂OH, —CH₂NR⁴₂, —CH₂CN, —CN, —C(S)NH₂, —OR³, —SR³, —N₃, —NHC(S)NR⁴₂, —NHAc, and a bond or A is absent;
each B and D are independently selected from —H, alkyl, alkenyl, alkynyl, aryl, alicyclic, aralkyl, alkoxyalkyl, —C(O)R¹¹, —C(O)SR³, —SO₂R¹¹, —S(O)R³, —CN, —NR⁹₂, —OR³, —SR³, perhaloalkyl, halo, —NO₂, and a bond, all except —H, —CN, perhaloalkyl, —NO₂, and halo are optionally substituted or each B and D are independently absent;
E is absent or is selected from —H, alkyl, alkenyl, alkynyl, aryl, alicyclic, alkoxyalkyl, —C(O)OR³, —CONR⁴₂, —CN, —NR⁹₂, —NO₂, —OR³, —SR³, perhaloalkyl, halo, and a bond, all except —H, —CN, perhaloalkyl, and halo are optionally substituted;
J is absent or is selected from —H and a bond;
X is an optionally substituted linking group that links R⁵ to the phosphorus atom via 2-4 atoms, including 0-1 heteroatoms selected from N, O, and S, except that if X is urea or carbamate there is 2 heteroatoms, measured by the shortest path between R⁵ and the phosphorus atom, and wherein the atom attached to the phosphorus is a carbon atom, and wherein there is no N in the linking group unless it is connected directly to a carbonyl or in the ring of a heterocycle; and wherein X is not a 2 carbon atom -alkyl- or -alkenyl- group; with the proviso that X is not substituted with —COOR², —SO₃R¹, or —PO₃R¹₂;
Y is independently selected from —O—, and —NR⁶—;
when Y is —O—, then R¹ attached to —O— is independently selected from —H, alkyl, optionally substituted aryl, optionally substituted alicyclic where the cyclic moiety contains a carbonate or thiocarbonate, optionally substituted -alkylaryl, —C(R²)₂OC(O)NR²₂, —NR²—C(O)—R³, —C(R²)₂—OC(O)R³, —C(R²)₂—O—C(O)OR³, —C(R²)₂OC(O)SR³, -alkyl-S—C(O)R³, -alkyl-S—S-alkylhydroxy, and -alkyl-S—S—S-alkylhydroxy,
when Y is —NR⁶—, then R¹ attached to —NR⁶— is independently selected from —H, —[C(R²)₂]_{q}—COOR³, —C(R⁴)₂COOR³, —[C(R²)₂]_{q}—C(O)SR, and -cycloalkylene-COOR³;
or when either Y is independently selected from —O— and —NR⁶—, then together R¹ and R¹ are -alkyl-S—S-alkyl- to form a cyclic group, or together R¹ and R¹ are 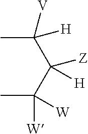
wherein
V, W, and W′ are independently selected from —H, alkyl, aralkyl, alicyclic, aryl, substituted aryl, heteroaryl, substituted heteroaryl, 1-alkenyl, and 1-alkynyl; or
together V and Z are connected via an additional 3-5 atoms to form a cyclic group containing 5-7 atoms, optionally 1 heteroatom, substituted with hydroxy, acyloxy, alkoxycarbonyloxy, or aryloxycarbonyloxy attached to a carbon atom that is three atoms from both Y groups attached to the phosphorus; or
together V and Z are connected via an additional 3-5 atoms to form a cyclic group, optionally containing 1 heteroatom, that is fused to an aryl group at the beta and gamma position to the Y attached to the phosphorus;
together V and W are connected via an additional 3 carbon atoms to form an optionally substituted cyclic group containing 6 carbon atoms and substituted with one substituent selected from hydroxy, acyloxy, alkoxycarbonyloxy, alkylthiocarbonyloxy, and aryloxycarbonyloxy, attached to one of said carbon atoms that is three atoms from a Y attached to the phosphorus;
together Z and W are connected via an additional 3-5 atoms to form a cyclic group, optionally containing one heteroatom, and V must be aryl, substituted aryl, heteroaryl, or substituted heteroaryl;
together W and W′ are connected via an additional 2-5 atoms to form a cyclic group, optionally containing 0-2 heteroatoms, and V must be aryl, substituted aryl, heteroaryl, or substituted heteroaryl;
Z is selected from —CHR²OH, —CHR²OC(O)R³, —CHR²OC(S)R³, —CHR²OC(S)OR³, —CHR²OC(O)SR³, —CHR²OCO₂R³, —OR², —SR², —CHR²N₃, —CH₂aryl, —CH(aryl)OH, —CH(CH═CR²₂)OH, —CH(C≡CR²)OH, —R², —NR²₂, —OCOR³, —OCO₂R³, —SCOR³, —SCO₂R³, —NHCOR², —NHCO₂R³, —CH₂NHaryl, —(CH₂)ₚ—OR², and —(CH₂)ₚ—SR²;
p is an integer 2 or 3;
q is an integer 1 or 2;
with the provisos that:
a) V, Z, W, W′ are not all —H; and
b) when Z is —R², then at least one of V, W, and W′ is not —H, alkyl, aralkyl, or alicyclic;
R² is selected from R³ and —H;
R³ is selected from alkyl, aryl, alicyclic, and aralkyl;
each R⁴ is independently selected from —H, and alkyl, or together R⁴ and R⁴ form a cyclic alkyl group;
R⁶ is selected from —H, lower alkyl, acyloxyalkyl, alkoxycarbonyloxyalkyl, and lower acyl;
each R⁹ is independently selected from —H, alkyl, aralkyl, and alicyclic, or together R⁹ and R⁹ form a cyclic alkyl group;
R¹¹ is selected from alkyl, aryl, —NR²₂, and —OR²; and
with the provisos that:
1) when G′ is N, then the respective A, B, D, or E is absent;
2) at least one of A and B, or A, B, D, and E is not —H or is not absent;
3) when R⁵ is a six-membered ring, then X is not any 2 atom linker, an optionally substituted -alkyl-, an optionally substituted -alkenyl-, an optionally substituted -alkyloxy-, or an optionally substituted -alkylthio-;
4) when G is N, then the respective A or B is not halogen or a group directly bonded to G via a heteroatom;
5) R¹ is not unsubstituted C1-C10 alkyl;
6) when X is not an -aryl- group, then R⁵ is not substituted with two or more aryl groups;
and pharmaceutically acceptable prodrugs and salts thereof.

4. The pharmaceutical composition of claim 3, wherein R⁵ is pyrrolyl, imidazolyl, oxazolyl, thiazolyl, isothiazolyl, 1,2,4-thiadiazolyl, pyrazolyl, isoxazolyl, 1,2,3-oxadiazolyl, 1,2,4-oxadiazolyl, 1,2,5-oxadiazolyl, 1,3,4-oxadiazolyl, 1,2,4-thiadiazolyl, 1,3,4-thiadiazolyl, 1,2,3-triazolyl, 1,2,4-triazolyl, 1,2,3,4-tetrazolyl, pyridinyl, pyrimidinyl, pyrazinyl, pyridazinyl, 1,3,5-triazinyl, 1,2,4-triazinyl, or 1,3-selenazolyl, all of which contain at least one substituent.

5. The pharmaceutical composition of claim 3, wherein R⁵ is selected from: 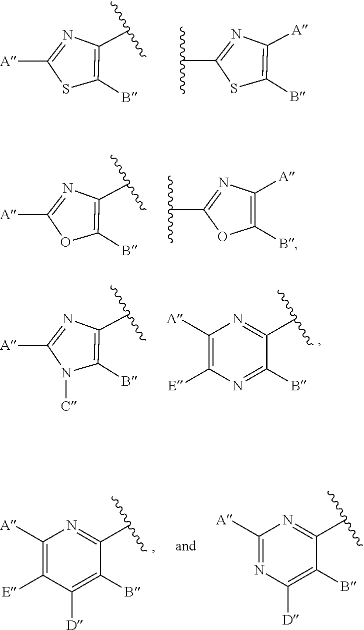
wherein
A″ is selected from —H, —NR⁴₂, —CONR⁴₂, —CO₂R³, halo, C1-C6 alkyl, C2-C6 alkenyl, C2-C6 alkynyl, C1-C6 perhaloalkyl, C1-C6 haloalkyl, aryl, —CH₂OH, —CH₂NR⁴₂, —CH₂CN, —CN, —C(S)NH₂, —OR³, —SR³, —N₃, —NHC(S)NR⁴₂, and —NHAc;
B″ and D″ are independently selected from —H, alkyl, alkenyl, alkynyl, aryl, alicyclic, aralkyl, alkoxyalkyl, —C(O)R¹¹, —C(O)SR³, —SO₂R¹¹, —S(O)R³, —CN, —NR⁹₂, —OR³, —SR³, perhaloalkyl, and halo, all except —H, —CN, perhaloalkyl, and halo are optionally substituted;
E″ is selected from —H, C1-C6 alkyl, C2-C6 alkenyl, C2-C6 alkynyl, aryl, C4-C6 alicyclic, alkoxyalkyl, —C(O)OR, —CONR⁴₂, —CN, —NR⁹₂, —OR³, —SR³, C1-C6 perhaloalkyl, and halo, all except —H, —CN, perhaloalkyl, and halo are optionally substituted; and
C″ is selected from —H, alkyl, alkylalkenyl, alkylalkynyl, aryl, alicyclic, aralkyl, aryloxyalkyl, and alkoxyalkyl, all optionally substituted;
R⁴ is selected from —H and C1-C2 alkyl; and
R¹¹ is selected from alkyl, aryl, —NR²₂, and —OR².

6. The pharmaceutical composition of claim 3, wherein X selected from -alkyl(hydroxy)-, -alkyl-, -alkynyl-, -aryl-, -carbonylalkyl-, -1,1-dihaloalkyl-, -alkoxyalkyl-, -alkyloxy-, -alkylthioalkyl-, -alkylthio-, -alkylaminocarbonyl-, -alkylcarbonylamino-, -alicyclic-, -aralkyl-, -alkylaryl-, -alkoxycarbonyl-, -carbonyloxyalkyl-, -alkoxycarbonylamino-, -alkylaminocarbonylamino-, -alkylamino-, and -alkenyl-, all optionally substituted.

7. The pharmaceutical composition of claim 6, wherein X is selected from -heteroaryl-, -alkylcarbonylamino-, -alkylaminocarbonyl-, -alkoxycarbonyl-, and -alkoxyalkyl-.

8. The pharmaceutical composition of claim 3, wherein R⁵ is 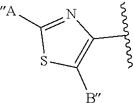
X is selected from methylenoxycarbonyl and furan-2,5-diyl; at least one Y group is —NH—; and pharmaceutically acceptable salts and prodrugs thereof.

9. The pharmaceutical composition of claim 3, wherein R⁵ is 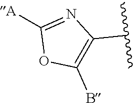
X is furan-2,5-diyl or methyleneoxycarbonyl, and A″ is —NH₂; at least one Y group is —NH—; and pharmaceutically acceptable salts and prodrugs thereof.

10. The pharmaceutical composition of claim 3, wherein the inhibitor is a compound of formula II or a salt thereof:

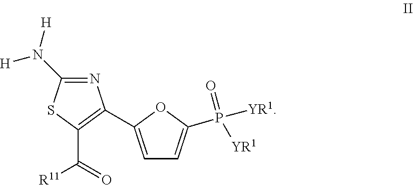

11. The pharmaceutical composition of claim 3, wherein the inhibitor is 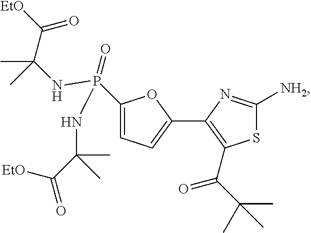 [SMILES: CCOC(=O)C(C)(C)NP(=O)(NC(C)(C)C(=O)OCC)c1ccc(-c2nc(N)sc2C(=O)C(C)(C)C)o1]
or a salt or prodrug thereof.

12. The pharmaceutical composition of claim 1, wherein the inhibitor is metformin.

13. The pharmaceutical composition of claim 1, wherein the agent is biotin, thiamine, riboflavin, coenzyme A or a salt, ester, or prodrug thereof.

14. A method to treat a metabolic condition, comprising administering the pharmaceutical composition of claim 1.

15. The method of claim 14, wherein the metabolic condition is diabetes.

16. The method of claim 15, wherein the diabetes is Type I, Type II, or gestational diabetes.

17. A method of treating a metabolic condition, comprising conjointly administering an inhibitor of gluconeogenesis or a salt, ester, or prodrug thereof and at least one agent that promotes lactic acid clearance, wherein the inhibitor and the agent, taken together, are therapeutically effective.

18. The method of claim 17, wherein the metabolic condition is diabetes.

19. The method of claim 18, wherein the diabetes is Type I, Type II, or gestational diabetes.
